(19) United States
(12) Patent Application Publication
(10) Pub. No.: US 2021/0220003 A1
(43) Pub. Date: Jul. 22, 2021

(54) TISSUE RESECTING INSTRUMENT

(71) Applicant: Covidien LP, Mansfield, MA (US)

(72) Inventor: Timothy J. Wood, Wilmington, MA (US)

(21) Appl. No.: 17/150,197

(22) Filed: Jan. 15, 2021

Related U.S. Application Data
(60) Provisional application No. 62/962,466, filed on Jan. 17, 2020.

(51) Int. Cl. A61B 17/32

(52) U.S. Cl. CPC A61B 17/32002; A61B 2017/320064; A61B 2017/320032

(57) ABSTRACT

An end effector assembly includes an outer shaft defining a longitudinal axis and a tapered distal end portion. A semi-spherical cutting implement is rotatably disposed in the outer shaft proximate the tapered distal end portion of the outer shaft. A portion of the rotating cutting implement protrudes from the tapered distal end portion of the outer shaft. The semi-spherical cutting implement includes a pinion gear. The pinion gear rotates the semi-spherical cutting implement about an axis substantially perpendicular to the longitudinal axis. An inner shaft is positioned within the outer shaft. The inner shaft defines a crown gear at a distal end portion thereof. The crown gear rotatably engage the pinion gear of the semi-spherical cutting implement. Rotation of the inner shaft about the longitudinal axis correspondingly rotates the pinon gear, which, in turn, rotates the semi-spherical cutting implement about the axis substantially perpendicular to the longitudinal axis.

### CROSS-REFERENCE TO RELATED APPLICATIONS

The present application claims the benefit of and priority to U.S. Patent Provisional Application No. 62/962,466, filed on Jan. 17, 2020, the entire content of which is incorporated herein by reference.

### FIELD

The present disclosure relates generally to the field of tissue resection. In particular, the present disclosure relates to a tissue resecting instrument configured to facilitate resection and removal of tissue from an internal surgical site, e.g., a uterus.

### BACKGROUND

Tissue resection may be performed endoscopically within an organ, such as a uterus, by inserting an endoscope (or hysteroscope) into the uterus and passing a tissue resection instrument through the endoscope (or hysteroscope) and into the uterus. With respect to such endoscopic tissue resection procedures, it often is desirable to distend the uterus with a fluid, for example, saline, sorbitol, or glycine. The inflow and outflow of the fluid during the procedure maintains the uterus in a distended state and flushes tissue and other debris from within the uterus to maintain a visible working space.

### SUMMARY

In accordance with an aspect of the present disclosure, an end effector assembly of a tissue-resecting device includes an outer shaft defining a longitudinal axis and a tapered distal end portion. A semi-spherical cutting implement is rotatably disposed in the outer shaft proximate the tapered distal end portion of the outer shaft. A portion of the rotating cutting implement protrudes from the tapered distal end portion of the outer shaft. The semi-spherical cutting implement includes a pinion gear. The pinion gear rotates the semi-spherical cutting implement about an axis substantially perpendicular to the longitudinal axis. An inner shaft is positioned within the outer shaft. The inner shaft defines a crown gear at a distal end portion thereof. The crown gear rotatably engage the pinion gear of the semi-spherical cutting implement. Rotation of the inner shaft about the longitudinal axis correspondingly rotates the pinon gear, which, in turn, rotates the semi-spherical cutting implement about the axis substantially perpendicular to the longitudinal axis.

In some aspects, the semi-spherical cutting implement includes a central hub supporting the pinion gear and a plurality of cutting edges extending axially from the central hub. The semi-spherical cutting implement defines a plurality of tissue capture windows between adjacent cutting edges of the plurality of cutting edges. The plurality of tissue capture windows pass cut tissue therethrough. The inner shaft is selectively advanceable between a proximal position in which the crown gear of the inner shaft is disengaged from the pinion gear of the semi-spherical cutting implement and a distal position in which the crown gear of the inner shaft is rotatably engaged with the pinion gear of the semi-spherical cutting implement.

In some aspects, the semi-spherical cutting implement includes a tissue capture reservoir defined below the plurality of tissue capture windows. The tissue capture reservoir captures cut tissue therein.

In some aspects, the outer shaft defines a central axis extending through a central region thereof. The inner shaft is spaced apart from the central axis of the outer shaft.

In some aspects, at least one cutting edge of the plurality of cutting edges defines a plurality of serrations thereon.

In some aspects, the semi-spherical cutting implement retracts into the outer shaft. The inner shaft translates between a proximal position in which the semi-spherical cutting implement is retracted into the outer shaft to conceal the distal portion of the semi-spherical cutting implement in the outer shaft and a distal positon in which a distal portion of the semi-spherical cutting implement protrudes from the outer shaft to cut tissue.

In accordance with an aspect of the present disclosure, a tissue-resecting device includes a handle and a longitudinal shaft extending from the handle. The longitudinal shaft supports the effector at a distal end portion thereof.

In some aspects, an outflow port applies suction to remove tissue from the tissue-resecting device toward the handle.

In some aspects, the end effector is detachable from the longitudinal shaft.

Other features of the disclosure will be appreciated from the following description.

### BRIEF DESCRIPTION OF THE DRAWINGS

The accompanying drawings, which are incorporated in and constitute a part of this specification, illustrate aspects and features of the disclosure and, together with the detailed description below, serve to further explain the disclosure, in which:

FIG. 1 is a side, perspective view of a tissue resecting instrument provided in accordance with aspects of the present disclosure;

FIG. 2 is top plan view of an end effector including a semi-spherical cutting implement in accordance with aspects of the present disclosure;

FIG. 3 is a top, perspective view of the semi-spherical cutting implement of FIG. 2;

FIG. 4 is a side view of the semi-spherical cutting implement of FIG. 2 illustrating a pinion gear of the semi-spherical cutting implement;

FIG. 5 is a side view of the semi-spherical cutting implement of FIG. 2 further illustrating an inner shaft defining a crown gear for rotatably engaging the pinion gear of the semi-spherical cutting implement; and

FIG. 6 is a side view of a semi-spherical cutting implement defining a plurality of serrations on a cutting edge thereof in accordance with aspects of the present disclosure.

### DETAILED DESCRIPTION

As used herein, the term “distal” refers to the portion that is being described which is further from a user, while the term “proximal” refers to the portion that is being described which is closer to a user. Further, to the extent consistent, any of the aspects and features detailed herein may be used in conjunction with any or all of the other aspects and features detailed herein.

As used herein, the terms parallel and perpendicular are understood to include relative configurations that are substantially parallel and substantially perpendicular up to about + or −10 degrees from true parallel and true perpendicular.

Exemplary axes or directions such as an X-axis direction, a Y-axis direction and a Z-axis direction may be illustrated in the accompanying drawings and/or described herein. As an example, the X-axis direction may perpendicular to the Y-axis direction, and the Z-axis direction may be orthogonal to the X-axis direction and the Y-axis direction.

“About” or “approximately” or “substantially” as used herein may be inclusive of the stated value and means within an acceptable range of variation for the particular value as determined by one of ordinary skill in the art, considering the measurement in question and the error associated with measurement of the particular quantity (e.g., the limitations of the measurement system). For example, “about” may mean within one or more standard variations, or within ±30%, 20%, 10%, 5% of the stated value.

Descriptions of technical features or aspects of an exemplary embodiment of the disclosure should typically be considered as available and applicable to other similar features or aspects in another exemplary embodiment of the disclosure. Accordingly, technical features described herein according to one exemplary embodiment of the disclosure may be applicable to other exemplary embodiments of the disclosure, and thus duplicative descriptions may be omitted herein.

Exemplary embodiments of the disclosure will be described more fully below (e.g., with reference to the accompanying drawings). Like reference numerals may refer to like elements throughout the specification and drawings.

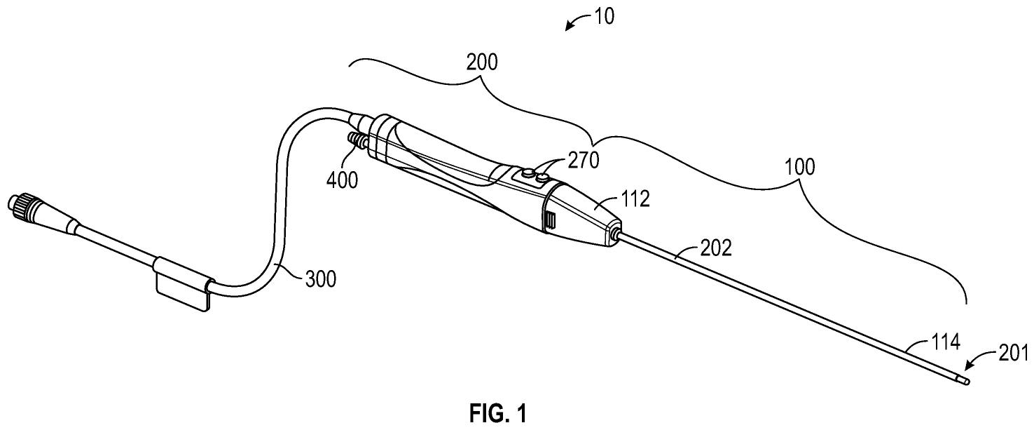

Referring generally to FIG. 1, a tissue resecting instrument 10 is provided in accordance with the present disclosure and is configured to resect tissue. Resecting instrument 10 includes a handle assembly 200, a longitudinal shaft 100 extending from the handle assembly 200, and an end effector assembly 201 supported at a distal end portion 114 of the longitudinal shaft 100. A proximal end portion 112 of the longitudinal shaft is coupled to the handle assembly 200. Tissue resecting instrument 10 is adapted to connect to a control unit (not shown) via a cable 300 to provide power and control functionality to tissue resecting instrument 10, although tissue resecting instrument 10 may alternatively or additionally include a power source, e.g., battery, and/or a control unit disposed within handle assembly 200. Tissue resecting instrument 10 is further adapted to connect to a fluid management system (not shown) via outflow tubing (see, e.g., outflow tubing 510) connected to outflow port 400 for applying suction to remove fluid, tissue, and debris from a surgical site via tissue resecting instrument 10. The control unit and fluid management system may be integral with one another, coupled to one another, or separate from one another.

Tissue resecting instrument 10 may be configured as a single-use device that is discarded after use or sent to a manufacturer for reprocessing, a reusable device capable of being cleaned and/or sterilized for repeated use by the end-user, or a partially-single-use, partially-reusable device. With respect to partially-single-use, partially-reusable configurations, handle assembly 200 may be configured as a cleanable/sterilizable, reusable component, while longitudinal shaft 100 is configured as a single-use, disposable/reprocessable component. In any of the above configurations, longitudinal shaft 100 is configured to releasably engage handle assembly 200 to facilitate disposal/reprocessing of any single-use components and cleaning and/or sterilization of any reusable components. Further, enabling releasable engagement of longitudinal shaft 100 with handle assembly 200 allows for interchangeable use of different longitudinal shafts support different end effector assemblies, e.g., different length, configuration, etc.

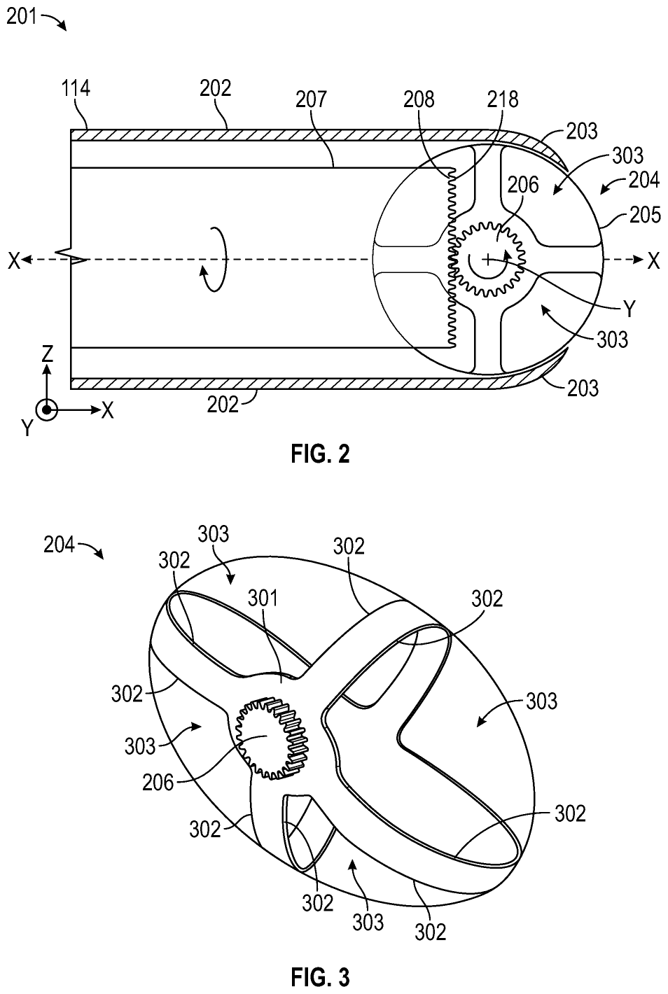

Referring to FIGS. 1 to 5, the end effector assembly 201 of the tissue-resecting device 10 includes an outer shaft 202 defining a longitudinal axis (see, e.g., axis X-X of FIG. 2) and a tapered distal end portion 203. A semi-spherical cutting implement 204 is rotatably disposed in the outer shaft 202 proximate the tapered distal end portion 203 of the outer shaft 202. A portion (see, e.g., FIGS. 2 and 5) of the semi-spherical cutting implement 204 protrudes from the tapered distal end portion 203 of the outer shaft 202.

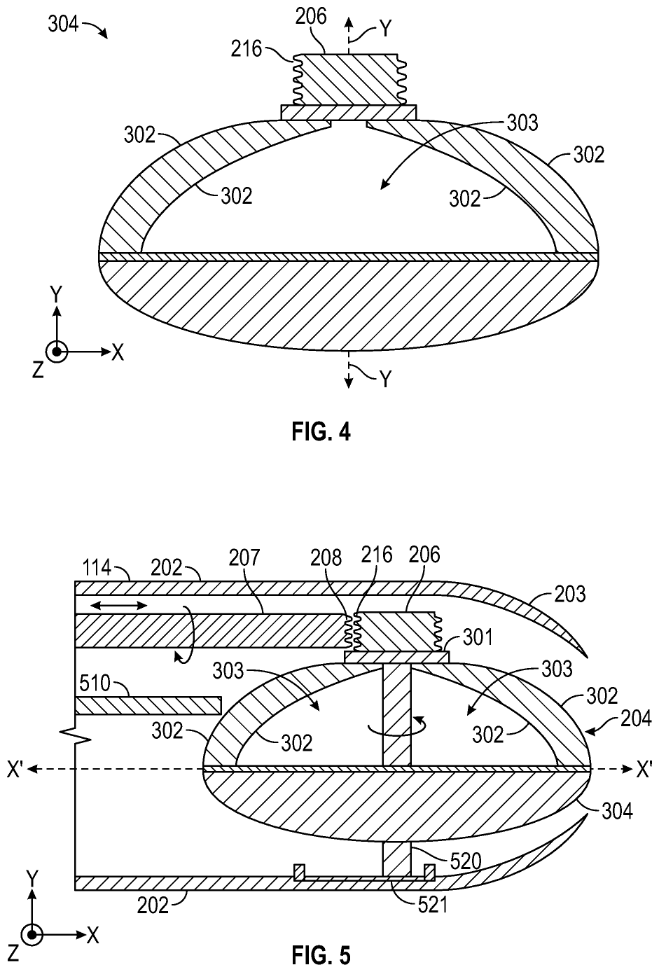

The semi-spherical cutting implement 204 includes a pinion gear 206. The pinion gear 206 defines a plurality of teeth 216. The pinion gear 206 rotates the semi-spherical cutting implement 204 about an axis (e.g., axis Y-Y of FIGS. 2 and 4) substantially perpendicular to the longitudinal axis (X-X). An inner shaft 207 is positioned within the outer shaft 202. The inner shaft 207 defines a crown gear 208 at a distal end portion thereof. The crown gear 208 rotatably engage the pinion gear 206 of the semi-spherical cutting implement 204. The crown gear 208 defines a plurality of teeth 218 configured to engage the teeth 216 of the pinion gear 206. Rotation of the inner shaft 207 about the longitudinal axis correspondingly rotates the pinon gear 206, which, in turn, rotates the semi-spherical cutting implement 204 about the axis substantially perpendicular to the longitudinal axis.

The semi-spherical cutting implement 204 includes a central hub 301 supporting the pinion gear 206 and a plurality of cutting edges 302 extending axially from the central hub 301. The semi-spherical cutting implement 204 defines a plurality of tissue capture windows 303 between adjacent cutting edges of the plurality of cutting edges 302. The plurality of tissue capture windows 303 pass cut tissue therethrough. The inner shaft 207 is selectively advanceable between a proximal position (see, e.g., FIG. 5) in which the crown gear 208 of the inner shaft 207 is disengaged from the pinion gear 206 of the semi-spherical cutting implement 204 and a distal position (see, e.g., FIG. 2) in which the crown gear 208 of the inner shaft 207 is rotatably engaged with the pinion gear 206 of the semi-spherical cutting implement 204 (e.g., by interlaying teeth 216 between teeth 218).

The semi-spherical cutting implement 204 includes a tissue capture reservoir 304 defined below the plurality of tissue capture windows 303. The tissue capture reservoir 304 captures cut tissue therein. The outflow tubing 510 connected to outflow port 400 may provide suction to remove captured tissue from the tissue capture reservoir 304.

The semi-spherical cutting implement 204 described herein may be replaced with a substantially spherical cutting implement, such as a completely spherical cutting implement.

The outer shaft 202 defines a central axis (see, e.g., axis X1-X1 of FIG. 5) extending through a central region thereof. The inner shaft 207 is spaced apart from the central axis of the outer shaft 202.

In use, the semi-spherical cutting implement 204 can be retracted into the outer shaft 202 to conceal the semi-spherical cutting implement 204 in the outer shaft 202. As desired, the semi-spherical cutting implement 204 can be deployed, at least partially, from the outer shaft 202 to expose a distal portion of the semi-spherical cutting implement 204. The inner shaft 207 translates between a proximal position in which the semi-spherical cutting implement 204 is retracted into the outer shaft 202 to conceal the distal portion of the semi-spherical cutting implement 204 in the outer shaft 202 and a distal positon in which a distal portion of the semi-spherical cutting implement 204 protrudes from the outer shaft 202 to cut tissue. That is, advancing the inner shaft 207 to contact the pinion gear 206 of the semi-spherical cutting implement 204 can force the semi-spherical cutting implement 204 into a more distal position to expose at least a portion of a distal-most end of the semi-spherical cutting implement 204.

Referring particularly to FIG. 5, central hub 301 may be supported to rotate about a shaft 520 secured to outer shaft 202. The shaft 520 may slide along a track 521 to allow proximal and distal advancement of the semi-spherical cutting implement 204, as described herein. The shaft 520 may be under tension (e.g., via a compression spring and/or an extension spring) to bias the shaft 520 into a more proximal position along the track 521 in which the semi-spherical cutting implement 204 is concealed within the outer shaft 202. Thus, distally advancing the inner shaft 207 to distally advance the semi-spherical cutting implement 204 may apply pressure against the biasing force of the shaft 520 under tension.

Handle assembly 200 further includes activation buttons 270 (see FIG. 1). One of activation buttons 270 advances inner shaft 207 to deploy the semi-spherical cutting implement 204 so that the distal portion of the semi-spherical cutting implement 204 protrudes from the outer shaft 203 to cut tissue. The other of activation buttons 270 activates rotation of inner shaft 207 to rotate crown gear 208, which, in turn, rotates the pinion gear 206, thus rotating the semi-spherical cutting implement 204 for cutting tissue.

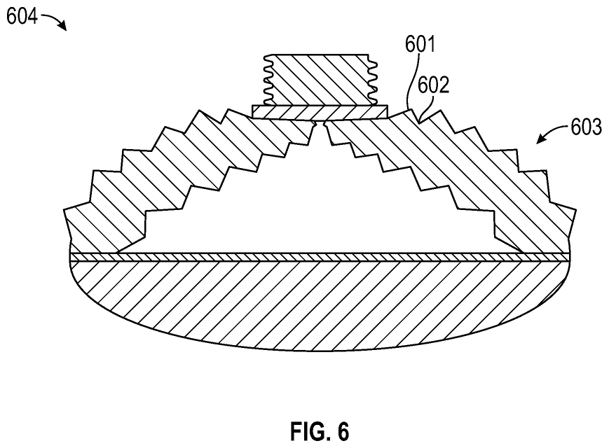

Referring to FIG. 6, a semi-spherical cutting implement 604 having at least one serrated edge 603 is illustrated. The semi-spherical cutting implement 604 is substantially the same as the semi-spherical cutting implement 204 described above, other than the serrated edges 603. At least one cutting edge of the plurality of cutting edges 602 defines a plurality of serrations 601 thereon. The serrations 601 may increase the cutting effectiveness of the semi-spherical cutting implement 604.

The various embodiments disclosed herein may also be configured to work with robotic surgical systems and what is commonly referred to as “Telesurgery.” Such systems employ various robotic elements to assist the surgeon and allow remote operation (or partial remote operation) of surgical instrumentation. Various robotic arms, gears, cams, pulleys, electric and mechanical motors, etc. may be employed for this purpose and may be designed with a robotic surgical system to assist the surgeon during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of surgeons or nurses may prep the patient for surgery and configure the robotic surgical system with one or more of the instruments disclosed herein while another surgeon (or group of surgeons) remotely controls the instruments via the robotic surgical system. As can be appreciated, a highly skilled surgeon may perform multiple operations in multiple locations without leaving his/her remote console which can be both economically advantageous and a benefit to the patient or a series of patients.

The robotic arms of the surgical system are typically coupled to a pair of master handles by a controller. The handles can be moved by the surgeon to produce a corresponding movement of the working ends of any type of surgical instrument (e.g., end effectors, graspers, knifes, scissors, etc.) which may complement the use of one or more of the embodiments described herein. The movement of the master handles may be scaled so that the working ends have a corresponding movement that is different, smaller or larger, than the movement performed by the operating hands of the surgeon. The scale factor or gearing ratio may be adjustable so that the operator can control the resolution of the working ends of the surgical instrument(s).

The master handles may include various sensors to provide feedback to the surgeon relating to various tissue parameters or conditions, e.g., tissue resistance due to manipulation, cutting or otherwise treating, pressure by the instrument onto the tissue, tissue temperature, tissue impedance, etc. As can be appreciated, such sensors provide the surgeon with enhanced tactile feedback simulating actual operating conditions. The master handles may also include a variety of different actuators for delicate tissue manipulation or treatment further enhancing the surgeon's ability to mimic actual operating conditions.

From the foregoing and with reference to the various figure drawings, those skilled in the art will appreciate that certain modifications can also be made to the disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. An end effector assembly of a tissue-resecting device, the end effector assembly comprising:
an outer shaft defining a longitudinal axis and a tapered distal end portion;
a semi-spherical cutting implement rotatably disposed in the outer shaft proximate the tapered distal end portion of the outer shaft, a portion of the rotating cutting implement configured to protrude from the tapered distal end portion of the outer shaft, the semi-spherical cutting implement including a pinion gear configured to rotate the semi-spherical cutting implement about an axis substantially perpendicular to the longitudinal axis; and
an inner shaft disposed within the outer shaft, the inner shaft defining a crown gear at a distal end portion thereof, the crown gear configured to rotatably engage the pinion gear of the semi-spherical cutting implement,
wherein rotation of the inner shaft about the longitudinal axis correspondingly rotates the pinon gear, which, in turn, rotates the semi-spherical cutting implement about the axis substantially perpendicular to the longitudinal axis.

2. The end effector of claim 1, wherein the semi-spherical cutting implement includes a central hub supporting the pinion gear and a plurality of cutting edges extending axially from the central hub.

3. The end effector of claim 2, wherein the semi-spherical cutting implement defines a plurality of tissue capture windows between adjacent cutting edges of the plurality of cutting edges, the plurality of tissue capture windows configured to pass cut tissue therethrough.

4. The end effector of claim 3, wherein the inner shaft is selectively advanceable between a proximal position wherein the crown gear of the inner shaft is disengaged from the pinion gear of the semi-spherical cutting implement and a distal position wherein the crown gear of the inner shaft is rotatably engaged with the pinion gear of the semi-spherical cutting implement

5. The end effector of claim 3, wherein the semi-spherical cutting implement includes a tissue capture reservoir defined below the plurality of tissue capture windows, the tissue capture reservoir configured to contain cut tissue therein.

6. The end effector of claim 1, wherein the outer shaft defines a central axis extending through a central region thereof, and wherein the inner shaft is spaced apart from the central axis of the outer shaft.

7. The end effector of claim 2, wherein at least one cutting edge of the plurality of cutting edges defines a plurality of serrations thereon.

8. The end effector of claim 1, wherein the semi-spherical cutting implement is configured to retract into the outer shaft.

9. The end effector of claim 8, wherein the inner shaft is configured to translate between a proximal position wherein the semi-spherical cutting implement is retracted into the outer shaft to conceal the distal portion of the semi-spherical cutting implement in the outer shaft and a distal positon wherein a distal portion of the semi-spherical cutting implement protrudes from the outer shaft to cut tissue.

10. A tissue-resecting device, comprising:
a handle; and
a longitudinal shaft extending from the handle, the longitudinal shaft supporting an effector at a distal end portion thereof, the end effector including:
an outer shaft defining a longitudinal axis and a tapered distal end portion;
a semi-spherical cutting implement rotatably disposed in the outer shaft proximate the tapered distal end portion of the outer shaft, a portion of the rotating cutting implement configured to protrude from the tapered distal end portion of the outer shaft, the semi-spherical cutting implement including a pinion gear configured to rotate the semi-spherical cutting implement about an axis substantially perpendicular to the longitudinal axis; and
an inner shaft disposed within the outer shaft, the inner shaft defining a crown gear at a distal end portion thereof, the crown gear configured to rotatably engage the pinion gear of the semi-spherical cutting implement,
wherein rotation of the inner shaft about the longitudinal axis correspondingly rotates the pinon gear, which, in turn, rotates the semi-spherical cutting implement about the axis substantially perpendicular to the longitudinal axis.

11. The tissue-resecting device of claim 10, wherein the semi-spherical cutting implement includes a central hub supporting the pinion gear and a plurality of cutting edges extending axially from the central hub.

12. The tissue-resecting device of claim 11, wherein the semi-spherical cutting implement defines a plurality of tissue capture windows between adjacent cutting edges of the plurality of cutting edges, the plurality of tissue capture windows configured to pass cut tissue therethrough.

13. The tissue-resecting device of claim 12, wherein the inner shaft is selectively advanceable between a proximal position wherein the crown gear of the inner shaft is disengaged from the pinion gear of the semi-spherical cutting implement and a distal position wherein the crown gear of the inner shaft is rotatably engaged with the pinion gear of the semi-spherical cutting implement.

14. The tissue-resecting device of claim 12, wherein the semi-spherical cutting implement includes a tissue capture reservoir defined below the plurality of tissue capture windows, the tissue capture reservoir configured to contain cut tissue therein.

15. The tissue-resecting device of claim 10, wherein the outer shaft defines a central axis extending through a central region thereof, and wherein the inner shaft is spaced apart from the central axis of the outer shaft.

16. The tissue-resecting device of claim 11, wherein at least one cutting edge of the plurality of cutting edges defines a plurality of serrations thereon.

17. The tissue-resecting device of claim 10, wherein the semi-spherical cutting implement is configured to retract into the outer shaft.

18. The tissue-resecting device of claim 17, wherein the inner shaft is configured to translate between a proximal position wherein the semi-spherical cutting implement is retracted into the outer shaft to conceal the distal portion of the semi-spherical cutting implement in the outer shaft and a distal positon wherein a distal portion of the semi-spherical cutting implement protrudes from the outer shaft to cut tissue.

19. The tissue-resecting device of claim 10, further comprising an outflow port configured to apply suction to remove tissue from the tissue-resecting device toward the handle.

20. The tissue-resecting device of claim 10, wherein the end effector is detachable from the longitudinal shaft.
